(19) United States
(12) Patent Application Publication
(10) Pub. No.: US 2024/0091290 A1
(43) Pub. Date: Mar. 21, 2024

(54) PHARMACEUTICAL COMPOSITION FOR RELIEVING PAIN FROM JOINT AND MUSCLE STRAIN

(71) Applicant: WEI ZHI OOI, AMPANG (MY)

(72) Inventor: WEI ZHI OOI, AMPANG (MY)

(21) Appl. No.: 18/164,641

(22) Filed: Feb. 6, 2023

(30) Foreign Application Priority Data
Sep. 15, 2022 (MY) PI2022005045

(51) Int. Cl. A61K 36/185; A61K 9/00; A61K 9/48; A61K 36/46; A61K 36/487; A61P 29/00

(52) U.S. Cl. CPC A61K 36/185; A61K 9/009; A61K 9/48; A61K 36/46; A61K 36/487; A61P 29/00

(57) ABSTRACT

A pharmaceutical composition that is effective in relieving pain from joint and muscle strain is disclosed herein. The pharmaceutical composition comprises a combination of Pecan kernels; Eucommia ulmoides barks; and Psoralea corylifolia seeds as it active ingredients.

### FIELD OF THE INVENTION

The present invention relates to a pharmaceutical composition. More particularly, the present invention relates to a pharmaceutical composition for relieving pain from joint and muscle strain.

### BACKGROUND ART

Pain is often understood to be a physical ailment or discomfort brought on by an accident or illness. The terms “chronic” and “acute” refer to different types of pain, depending on how long they continue. Chronic pain is described as persisting for a long time, whereas acute pain ends rapidly. Although muscle aches and pains may occur due to tension, over-activity, or injury, many different conditions affect the muscles and surrounding tissues, including the connective tissues, ligaments and tendons. Injuries to the joints and circumstances that set off inflammation are two reasons why joint discomfort might develop. These two symptoms frequently occur in tandem because muscles combine at the joints. In most cases, joint problems do not result in fatalities. However, it is believed that these illnesses have the biggest influence on people's quality of life, resulting in impairment, loss of independence, fewer social interactions, distress, and diminished well-being for those who have these kinds of illnesses.

Hot and cold packs, topical and oral analgesics, non-steroidal anti-inflammatory drugs (NSAIDs), stronger medications like muscle relaxants, anti-anxiety medications, and antidepressants, prescription NSAIDs, steroidal injections at the site of joint swelling/inflammation (in cases of chronic pain cases), as well as invasive and non-invasive treatments, have all been used as traditional forms of these aforementioned treatments. However, these non-steroidal anti-inflammatory drugs (NSAIDs) must be taken for extended periods of time in order to diminish both the discomfort. Despite being among the most commonly used medications, these ones typically cause gastrointestinal (GI) ulcers, indigestion, burning, and bleeding as side effects (Wallace and Vong, 2008).

Numerous people are now turning to herbal remedies, phytonutrients, and nutraceuticals for the treatment of a variety of health issues in various national healthcare settings as the use of these products continues to grow quickly throughout the world (WHO, 2004). Natural therapies have undoubtedly gained enormous popularity over the past ten years, both in developed and emerging nations, as seen by the availability of these herbal cures not only in drugstores but also in food and grocery stores. Up to four billion people, or 80% of the world's population, are thought to rely on herbal medicines as their main source of healthcare, and the use of herbs in traditional medicine is seen as an essential part of this practice.

Essentially, herbal treatments are made up of fragments of plants or unpurified plant extracts that include a variety of ingredients that interact in a synergistic way. The U.S. Pat. No. 10,583,120, for instance, discloses a pharmaceutical composition or dietary supplement for the treatment of muscle, bone, articular, or tendon diseases that has a muscle-relaxant and anti-inflammatory activity. This composition or supplement is useful in the treatment of diseases of the muscles, bones, joints, and tendons and contains at least one ketoboswellic acid from Boswellia serrata dry extract, magnesium, and L-tryptophan as active components. Another US patent publication, 20150024075, describes a phytocomposition that contains the following extracts: (a) 0.01 weight percent to 26 weight percent of Arnica montana extract; (b) 0.01 weight percent to 26 weight percent of Rosmarinus officinalis extract; (c) 0.01 weight percent to 26 weight percent of Zingiber officinale extract; and (d) 0.01 weight percent to 26 weight percent of Boswellia serrate extract. This phytocomposition can be combined with a pharmaceutically acceptable carrier to create a pharmaceutical composition that can be used to treat muscle, arthritic, and rheumatic pain in patients who have joint diseases, in particular rheumatoid arthritis, osteoarthritis, fibromyalgia, gouty arthritis, psoriatic arthritis, lupus, and juvenile arthritis.

In light of the limitations of non-steroidal anti-inflammatory medicines (NSAIDs) and the potential benefit of these herbal remedies in the partial remission of the initial symptom, therefore, it is an object of the invention to provide a synergistic composition that uses plant fragments, unpurified plant extracts, or purified plant extracts to relieve pain from joint and muscular strain.

### SUMMARY OF THE INVENTION

An object of the present invention is to provide a pharmaceutical composition for relieving pain from muscle and joint strain comprising:
a) Pecan kernels
b) Eucommia ulmoides barks; and
c) Psoralea corylifolia seeds.

Another object of the present invention is to provide the pharmaceutical composition, wherein ratio of said pecan kernels to said Eucommia ulmoides barks to said Psoralea corylifolia seeds is 1:2:2.

Another object of the present invention is to provide the pharmaceutical composition, wherein said Eucommia ulmoides barks and said Psoralea corylifolia seeds are heated Eucommia ulmoides barks and heated Psoralea corylifolia seeds.

Another object of the present invention is to provide the pharmaceutical composition, wherein said pharmaceutical composition further comprising water.

Another object of the present invention is to provide the pharmaceutical composition, wherein said pharmaceutical composition further comprising sweetener.

Another object of the present invention is to provide the pharmaceutical composition, wherein said pharmaceutical composition is an oral composition.

Another object of the present invention is to provide the pharmaceutical composition, wherein said pharmaceutical composition comprises a solid dosage form or a liquid dosage form.

Another object of the present invention is to provide the pharmaceutical composition, wherein said solid dosage form is selected from a capsule or a trilayer tablet.

Another object of the present invention is to provide the pharmaceutical composition, wherein said liquid dosage form is kept in a sachet.

Another object of the present invention is to provide the pharmaceutical composition, wherein said pharmaceutical composition further comprising walnut kernels.

### DETAILED DESCRIPTION

A pharmaceutical composition for relieving pain from muscle and joint strain is disclosed herein. Hereinafter, this specification will describe the present invention according to the preferred embodiments of the present invention. However, it is to be understood that limiting to the preferred embodiments of the invention is merely to facilitate discussion of the present invention and envisioned that those skilled in the art may devise various modifications and equivalents without departing from the scope of the appended claims.

As used in this specification and the appended claims, the singular forms “a”, “an”, and “the” encompass embodiments having plural referents, unless the content clearly dictates otherwise. As used in this specification, use of a singular form of a term, can encompass embodiments including more than one of such term, unless the content clearly dictates otherwise. As used in this specification and the appended claims, the term “or” is generally employed in its sense including “either or both” unless the context clearly dictates otherwise.

As used herein, the term “about” modifying the quantity of an ingredient of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” as used herein refers to a range of values ±0.5% of a specified value. For example, the phrase “about 177 microns” includes ±0.5% of 177 microns, or from 176.1 to 177.9 microns.

The invention will now be described in detail in connection with certain preferred and optional embodiments, so that various aspects thereof may be more fully understood and appreciated.

The composition that is the subject of the present invention is characterized in that it comprises, as active ingredients, a combination of pecan kernels, Eucommia ulmoides barks; and Psoralea corylifolia seeds. In one embodiment, the composition that is the subject of the present invention comprises a combination of pecan kernels, Eucommia ulmoides barks, and Psoralea corylifolia seeds in a ratio of pecan kernels to Eucommia ulmoides barks to Psoralea corylifolia seeds of 1:2:2. In one embodiment, the Eucommia ulmoides barks and Psoralea corylifolia seeds are preferably a heated Eucommia ulmoides barks and a heated Psoralea corylifolia seeds. This could improve the quality and reduce the moisture of the ingredients. In another alternative embodiment, if desired, the composition that is subject to the present invention can comprise, as active ingredients, a combination of a mixture of walnut kernels and pecan kernels; Eucommia ulmoides barks; and Psoralea corylifolia seeds. Herein, the ratio of the mixture of walnut kernels and pecan kernels to Eucommia ulmoides barks to Psoralea corylifolia seeds is preferably 1:2:2. In one embodiment, the Eucommia ulmoides barks and Psoralea corylifolia seeds are preferably a heated Eucommia ulmoides barks and a heated Psoralea corylifolia seeds. This could improve the quality and reduce the moisture of the ingredients.

The composition that is the subject of the present invention is intended to reduce pain from strained joints and muscles as well as ligaments discomfort caused by improper or poor posture when using a computer or a phone without having to rely solely on non-steroidal anti-inflammatory drugs (NSAIDs). Eucommia ulmoides barks have been used in eastern medicine for centuries, and it is believed that they are beneficial for the health of the muscles and joints. Psoralea corylifolia L. (Leguminosae) is a well-known traditional medicinal plant used since ancient times for the treatment of various ailments including for the treatment of bone fractures and also for joints. Pecan kernels have been shown to have important health benefits to humans due to their lipid constituents and phenolic compounds. These phenolic compounds have been proven to be beneficial against several cancers and inflammation, and to have antiviral and antihypertensive activity, among others. Alternatively, walnut kernels, which are rich in various anti-inflammatory elements like omega-3 fatty acids, can assist in alleviating painful inflammation in the joints and connective tissue. Therefore, the combination of the abovementioned active ingredients in the composition according to the invention is particularly advantageous since they develop a synergistic effect with regard to the muscle- and joint-relaxant action by delivering quick and effective pain relief for painful joints and muscles.

The composition of the present invention may be formulated in any form suitable for oral administration. Preferably, the composition is formulated as an oral composition in a solid dosage form or a liquid dosage form. Herein, the solid dosage form is preferably selected from a capsule or a tablet. The capsule may be a soft or hard gelatin capsule. The tablet is more preferably a trilayer tablet. The liquid dosage form is preferably kept in a sachet.

In another embodiment, if desired, the composition that is subject to the present invention can comprise one or more pharmaceutically acceptable excipients. The term “excipient” herein refers to any substance that is not a therapeutic agent but is utilized as a carrier or vehicle for delivery of a therapeutic agent to a subject or added to a pharmaceutical composition to improve its handling or storage properties or to permit or facilitate the formation of a dose unit of the composition. Excipients include, by way of illustration and not limitation, diluents, disintegrants, binding agents, adhesives, wetting agents, lubricants, glidants, surface modifying agents, substances added to mask or counteract a disagreeable taste or odor, flavors, dyes, fragrances, and substances added to improve the appearance of the composition. In accordance with the disclosure in the present study, any such excipients may be utilized in any dosage form. Excipients optionally employed in the composition that is subject to the present invention can be solids, semi-solids, liquids, or combinations thereof. The composition that is subject to the present invention containing excipients can be prepared by any known technique of pharmacy that comprises admixing an excipient with a drug or therapeutic agent. In another embodiment, the composition that is subject to the present invention optionally comprises water as an excipient, particularly for the liquid dosage form.

The composition that is subject to the present invention can comprise one or more binding agents or adhesives as excipients, particularly for the solid dosage form. Such binding agents and adhesives may impart sufficient cohesion to the powder being tableted to allow for normal processing operations such as sizing, lubrication, compression, and packaging, but still, allow the tablet to disintegrate and the composition to be absorbed upon ingestion. Suitable binding agents and adhesives that can be used in the present embodiments include, either individually or in combination, by way of example, and are not limited to sucrose, gelatin, glucose, starch, or the like.

The composition that is subject to the present invention can comprise one or more sweeteners, particularly for the liquid dosage form. Sweeteners that can be used in the present embodiments include, by way of example, and are not limited to aspartame, cyclamate, dextrose, isomalt, maltitol, mannitol, saccharin, sorbitol, stevia, xylitol, or the like.

The composition of the present invention may be formulated in a manner suitable for oral administration and will be prepared according to the conventional method that is well known in the pharmaceutical field. For example, compositions in solid dosage forms can be prepared by processes that employ one or a combination of methods including, without limitation, (1) dry mixing, (2) direct compression, (3) milling, (4) dry or non-aqueous granulation, (5) wet granulation, or (6) fusion. A particularly preferred formulation of the composition that is subject to the present invention, used in the experimental studies, is further depicted in the illustration of the following example which should not be construed as limiting. In addition to the indicated active ingredients, the formulation comprises suitable excipients and optionally flavourings or colourants. The advantages of the present invention are also further depicted herein.

The following example is illustrative only, and are not intended to limit, in any manner, any of the aspects described herein.

### Example 1

### Capsule Of The Composition Of The Present Invention And Its Effectiveness In Relieving Pain From Muscle And Joint Strain

A. Capsule Formulation

Pecan kernels, Eucommia ulmoides barks, and Psoralea corylifolia seeds were combined in a 1:2:2 ratio to form a capsule of the composition that is the subject of the present invention (hereinafter denoted as Capsule A) for relieving pain from muscle and joint strain. The following Table 1 showed the capsule formulation of the composition that is subject to the present invention.

TABLE 1
Capsule formulation of the composition that
is subject to the present invention

| Component Capsule A | Amount |
|---|---|
| Pecan kernels | 50 g |
| Eucommia ulmoides barks | 100 g |
| Psoralea corylifolia seeds | 100 g |

Six additional capsules (hereinafter denoted as capsule B; capsule C; capsule D; capsule E; capsule F and capsule G) were also prepared as a comparison. These capsules employed the same ratio, but instead of pecan kernels, walnut kernels, hazelnut, pistachio, cashew, almond nut, and peanut kernels from the same family of nuts (Juglandaceae) were used. The following Table 2 showed the capsule formulation using different nuts from the Juglandaceae family.

TABLE 2
Capsule formulation using different nuts from Juglandaceae family

| Capsule | Component | Amount | |
|---|---|---|---|
| B | Walnut kernels | 50 | g |
| | Eucommia ulmoides barks | 100 | g |
| | Psoralea corylifolia seeds | 100 | g |
| C | Hazelnut | 50 | g |
| | Eucommia ulmoides barks | 100 | g |
| | Psoralea corylifolia seeds | 100 | g |
| D | Pistachio nut | 50 | g |
| | Eucommia ulmoides barks | 100 | g |
| | Psoralea corylifolia barks | 100 | g |
| E | Cashew nut | 50 | g |
| | Eucommia ulmoides barks | 100 | g |
| | Psoralea corylifolia barks | 100 | g |
| F | Peanut | 50 | g |
| | Eucommia ulmoides barks | 100 | g |
| | Psoralea corylifolia barks | 100 | g |
| G | Almond nut | 50 | g |
| | Eucommia ulmoides barks | 100 | g |
| | Psoralea corylifolia barks | 100 | g |

B. Method of Preparing Capsules A through G

Capsule A was prepared in accordance with the formulation as described in the preceding Table 1. Firstly, 100 g of Eucommia ulmoides barks were heated for 15 minutes at a temperature of 180° C. After that, the heated Eucommia ulmoides barks were subjected to a grinding process to produce Eucommia ulmoides barks in powder form. The Eucommia ulmoides barks were heated and ground repeatedly until they were reduced to a powder with a particle size of about 177 microns (80 mesh). Subsequently, 100 g of Psoralea corylifolia seeds were also heated for 15 minutes at a temperature of 180° C. Then, the heated Psoralea corylifolia seeds were subjected to a grinding process to produce Psoralea corylifolia seeds in powder form. The Psoralea corylifolia seeds were heated and ground repeatedly until they were reduced to a powder with a particle size of about 177 microns (80 mesh). Then, pecan kernels were ground into a powder with a particle size of about 177 microns (80 mesh). Finally, the powdered Eucommia ulmoides bark, Psoralea corylifolia seeds, and pecan kernels were combined into a capsule and ready for consumption. This procedure was repeated to prepare the additional six capsules B through G, using the formulation listed in the preceding Table 2.

C. The Effectiveness Of Capsules A Through G In Relieving Pain From Muscle And Joint Strain

A human pilot study was carried out on subjects with strained muscles and joints. Each subject was given a different capsule, labelled A through G, each containing 280 mg of the active ingredient, to be taken once daily. They were also observed and monitored. The average of effectiveness sign of capsules A through G in reducing pain from muscle and joint strain was shown in the following Table 3.

TABLE 3
Average of effectiveness sign after consuming capsules A
through G in relieving pain from muscle and joint strain

| Capsule | Average of effectiveness sign (hours) |
|---|---|
| A | about 3 hours and 45 minutes |
| B | about 4 hours |
| C | not effective |
| D | not effective |
| E | not effective |
| F | not effective |
| G | not effective |

By referring to the aforementioned Table 3, it can be seen that capsule A, which was prepared using the formulation of the composition of the present invention, demonstrated the fastest and most effective results in relieving pain from muscle and joint strain. Another option is Capsule B, which has walnut kernels as one of its active ingredients and has been shown to be useful in relieving pain from muscle and joint strain. Capsules C through G, which comprise hazelnut, pistachio nut, cashew nut, peanut, and almond nut, was found to be ineffective in relieving pain from strained muscles and joints. These findings have demonstrated the value of including pecan kernels as one of the active ingredients in the composition of the present invention for reducing pain from strained muscles and joints. These results have also demonstrated the significance of using walnut kernels as one of the additional active ingredients in the composition of the present invention for relieving pain from muscle and joint strain.

### Example 2

### Liquid Formulation Of The Composition Of The Present Invention And Its Effectiveness In Relieving Pain From Muscle And Joint Strain

A. Liquid Formulation

Pecan kernels, Eucommia ulmoides barks, and Psoralea corylifolia seeds were combined in a ratio of 1:2:2 with water as an excipient and sweetener to make a liquid formulation of the composition that is the subject of the present invention for relieving pain from muscle and joint tension. This formulation was then stored in a 15 mL sachet (hereinafter denoted as sachet A). The following Table 4 showed the liquid formulation of the composition that is subject to the present invention.

TABLE 4
Liquid formulation of the composition that
is subject to the present invention

| Component Sachet A | Amount | |
|---|---|---|
| Pecan kernels | 40 | g |
| Eucommia ulmoides barks | 80 | g |
| Psoralea corylifolia seeds | 80 | g |
| Water | 1 | L |
| sweetener | 100 | mL |

Six additional liquid formulations (hereinafter denoted as sachet B; sachet C; sachet D; sachet E; sachet F and sachet G) were also prepared as a comparison. These liquid formulations employed the same ratio, but instead of pecan kernels, walnut kernels, hazelnut, pistachio, cashew, almond nut, and peanut kernels from the same family of nuts (Juglandaceae) were used. The following Table 5 showed the liquid formulation using different nuts from the Juglandaceae family.

TABLE 5
Liquid formulation using different nuts from Juglandaceae family

| Sachet | Component | Amount | |
|---|---|---|---|
| B | Walnut kernels | 50 | g |
| | Eucommia ulmoides barks | 100 | g |
| | Psoralea corylifolia seeds | 100 | g |
| | Water | 1 | L |
| | Sweetener | 100 | mL |
| C | Hazelnut | 50 | g |
| | Eucommia ulmoides barks | 100 | g |
| | Psoralea corylifolia seeds | 100 | g |
| | Water | 1 | L |
| | Sweetener | 100 | mL |
| D | Pistachio nut | 50 | g |
| | Eucommia ulmoides barks | 100 | g |
| | Psoralea corylifolia seeds | 100 | g |
| | Water | 1 | L |
| | Sweetener | 100 | mL |
| E | Cashew nut | 50 | g |
| | Eucommia ulmoides barks | 100 | g |
| | Psoralea corylifolia seeds | 100 | g |
| | Water | 1 | L |
| | Sweetener | 100 | mL |
| F | Peanut | 50 | g |
| | Eucommia ulmoides barks | 100 | g |
| | Psoralea corylifolia seeds | 100 | g |
| | Water | 1 | L |
| | Sweetener | 100 | mL |
| G | Almond nut | 50 | g |
| | Eucommia ulmoides barks | 100 | g |
| | Psoralea corylifolia seeds | 100 | g |
| | Water | 1 | L |
| | Sweetener | 100 | mL |

B. Method of Preparing Liquid Formulations (Sachet A through G)

Sachet A was prepared in accordance with the formulation as described in the preceding Table 4. Firstly, 1 L of water was placed in a container. Then, 40 g of pecan nut, 80 g of Eucommia ulmoides barks and 80 g of Psoralea corylifolia seeds were added to the water to form a mixture. The mixture was heated, with or without stirring, until the water content reached 500 mL. To speed up the reduction process, stirring was done while the mixture was heated. The reduction of the water will take about 1.5 hours if stirring process is not performed. This procedure was repeated to prepare the additional six liquid formulations (sachet B through G), using the formulation listed in the preceding Table 5.

C. The Effectiveness Of Liquid Formulations (Sachet A Through G) In Relieving Pain From Muscle And Joint Strain

A human pilot study was carried out on subjects with strained muscles and joints. Each subject was given different liquid formulations (each in a 15 mL sachet, labelled A through G), to be taken once daily. They were also observed and monitored. The average of effectiveness sign of the liquid formulations in reducing pain from muscle and joint strain was shown in the following Table 6.

TABLE 6
Average of effectiveness sign after consuming the liquid formulations
in relieving pain from muscle and joint strain

| Sachet | Average of effectiveness sign (hours) |
|---|---|
| A | about 3 hours and 15 minutes |
| B | about 3 hours and 30 minutes |
| C | not effective |
| D | not effective |
| E | not effective |
| F | not effective |
| G | not effective |

By referring to the aforementioned Table 6, it can be seen that the liquid formulation that was kept in sachet A, which was prepared using the formulation of the composition of the present invention, demonstrated the fastest and most effective results in relieving pain from muscle and joint strain. Another option is the liquid formulation that was kept in sachet B, which has walnut kernels as one of its active ingredients and has been shown to be useful in relieving pain from muscle and joint strain. The liquid formulation that was kept in sachet C through G, which comprise hazelnut, pistachio nut, cashew nut, peanut, and almond nut, was found to be ineffective in relieving pain from strained muscles and joints. These findings have demonstrated the value of including pecan kernels as one of the active ingredients in the composition of the present invention for relieving pain from muscle and joint strain. These outcomes further highlight the value of including walnut kernels as one of the additional active ingredients in the composition of the present invention for relieving pain from muscle and joint strain.

While a particular example of the present invention has been shown and described, it is apparent that changes and modifications may be made therein without departing from the invention in its broadest aspect. The aim of the appended claims, thereof, is to cover all such changes and modifications which fall within the scope of the invention.

1. A pharmaceutical composition for relieving pain from muscle and joint strain comprising:
a) Pecan kernels
b) Eucommia ulmoides barks; and
c) Psoralea corylifolia seeds.

2. The pharmaceutical composition as claimed in claim 1, wherein ratio of said pecan kernels to said Eucommia ulmoides barks to said Psoralea corylifolia seeds is 1:2:2.

3. The pharmaceutical composition as claimed in claim 1, wherein said Eucommia ulmoides barks and said Psoralea corylifolia seeds are heated Eucommia ulmoides barks and heated Psoralea corylifolia seeds.

4. The pharmaceutical composition as claimed in claim 1, wherein said pharmaceutical composition further comprising water.

5. The pharmaceutical composition as claimed in claim 4, wherein said pharmaceutical composition further comprising sweetener.

6. The pharmaceutical composition as claimed in claim 1, wherein said pharmaceutical composition is an oral composition.

7. The pharmaceutical composition as claimed in claim 1, wherein said pharmaceutical composition comprises a solid dosage form or a liquid dosage form.

8. The pharmaceutical composition as claimed in claim 7, wherein said solid dosage form is selected from a capsule or a trilayer tablet.

9. The pharmaceutical composition as claimed in claim 7, wherein said liquid dosage form is kept in a sachet.

10. The pharmaceutical composition as claimed in claim 1, wherein said pharmaceutical composition further comprising walnut kernels.
